(19) United States
(12) Patent Application Publication
(10) Pub. No.: US 2025/0001134 A1
(43) Pub. Date: Jan. 2, 2025

(54) SINGLE-PART DEFLECTABLE CATHETER DEVICES AND METHODS FOR MAKING THEM

(71) Applicants: Stephen A. Leeflang, Sandy, UT (US); Jordan Johnson, West Valley City, UT (US); Christian S. Eversull, Palo Alto, CA (US)

(72) Inventors: Stephen A. Leeflang, Sandy, UT (US); Jordan Johnson, West Valley City, UT (US); Christian S. Eversull, Palo Alto, CA (US)

(21) Appl. No.: 18/652,793

(22) Filed: May 1, 2024

Related U.S. Application Data
(60) Provisional application No. 63/463,284, filed on May 1, 2023.
(60) Provisional application No. 63/463,285, filed on May 1, 2023.

(51) Int. Cl. A61M 25/01; A61M 25/00

(52) U.S. Cl. CPC A61M 25/0144; A61M 25/0053; A61M 25/0136

(57) ABSTRACT

Catheters, sheaths, or other tubular devices are provided, e.g., steerable tubular devices, including braided or other reinforcement configurations and/or including one or more steering elements for deflecting a distal portion, and/or to methods for making such catheters, sheaths, or other tubular devices. The devices and methods described herein may involve simplified, e.g., single-piece, construction of catheter shafts, which may save space and/or provide more robust construction of the resulting devices compared to conventional manufacturing methods.

### RELATED APPLICATION DATA

The present application claims benefit of co-pending U.S. provisional applications Ser. Nos. 63/463,284 and 63/463,285, filed May 1, 2023, the entire disclosures of which are expressly incorporated by reference herein.

### TECHNICAL FIELD

The present application relates generally to catheters, sheaths, or other tubular devices, and, more particularly, to tubular bodies for catheters, sheaths, or other tubular devices including braided or other reinforcement configurations and/or including one or more steering elements for deflecting a distal portion of the tubular devices, and to methods for making such tubular bodies and devices.

### BACKGROUND

Elongate tubular devices, such as diagnostic or treatment catheters or sheaths may be provided for introduction into a patient's body, e.g., the patient's vasculature or other body lumens. For example, a catheter may have a distal portion configured to be introduced into a body lumen and advanced to one or more desired locations within the patient's body by manipulating a proximal end of the catheter.

To facilitate introduction of such a catheter, one or more wires, cables, or other steering elements may be provided within the catheter, e.g., that are coupled to the distal portion and may be pulled or advanced from the proximal end to deflect the distal portion. For example, a steering element may be provided that is intended to deflect the distal portion within a predetermined plane and/or into a desired curved shape.

Pull wires are a common way to impart deflection ability to such a catheter. However, there are a number of drawbacks associated with such pull wires. For example, a pull wire occupies a significant amount of space within the catheter body. In addition, a pull wire frequently needs to be reinforced, e.g., on the inside and outside of the braid or other reinforcement of the catheter, e.g., to prevent “pull through” or loosening when the pull wire is actuated by pushing or pulling, i.e., the resulting bending moment may cause the pull wire to separate layers of or tear at least partially through the wall of catheter, potentially splitting the catheter and/or decreasing the mechanical actuation ability of the pull wire. Further, a pull wire can make the torque properties of the catheter non-homogenous, making it difficult or impossible to torque the catheter when the pull wire is actuated, e.g., under tension within a tortuous pathway. Further, auxiliary lumens, in particular those located in the wall of a large bore sheath, may be difficult to manufacture with consistency due to difficulties with alignment, hand assembly, and the like.

Pull and push wire based deflection mechanisms in catheters also create a number of design and performance challenges. These challenges may include but are not limited to 1) avoiding undesirable bending/deflection/waviness outside of the desired deflection area, 2) avoiding stiffening of the catheter greater than is desirable, 3) avoiding limitation on torque transmission to the distal portion of the device, 4) avoiding high deflection forces, and/or 5) achieving manufacturing flexibility for the position of the deflection segment.

Deflectable electrophysiology (“EP”) and other catheter shafts are almost universally constructed in at least two separate sections, sometimes more. The end objective of the shaft assembly is to create a highly torque responsive (smooth and responsive, even around a bend) single lumen proximal shaft portion and a multi-lumen distal portion to enable deflection via interaction with compression coils and pull wires in the peripheral lumens and one or more central lumens where other elements (conductors, irrigation, sensors, activation wires, etc.) must also pass. In order to maximize the torque properties of the proximal shaft, a single lumen construction that provides a uniform wall (and hence isometric bend and rotation properties) is preferable to a multi-lumen construction along the whole length. There are two typical state of the art constructions in EP catheter shafts to accomplish this: 1) a multi-lumen segment is bonded to the end of the single lumen section via a range of different bonding mechanism and 2) a multi-lumen segment is slid inside the distal portion of the single lumen shaft. Both constructions are then terminated at the distal end with elements more customized to the specific purpose of the catheter whether it be mapping, ablation, injection, sensing, etc., or combinations of all or some. Regardless of the customization, the same core shaft constructions described prevail effectively universally amongst all different varieties of actual deflectable EP catheters.

Problems with shafts for steerable devices generally fall into two general categories, namely manufacturing and design/performance. Multipart constructions are more complex and require additional steps that, even beyond the added labor, are also subject to yield problems. For example, the braid wire at the end of a single lumen portion of a shaft is difficult to prevent coming out during attachment of a multi-lumen section during heat welding (much preferred to adhesive bonding even in spite of the yield problem and additional manufacturing step). This same problem also creates a performance problem as the discontinuity in the braid itself weakens torque transmission, kink resistance, and general device integrity. Even further, some total wall thickness cross-section must be dedicated to this bond section to make it robust and, as such, limits the amount of available space left for other key elements that must pass through. As EP and other catheters continue to evolve with more and more sensors, effectors and other mechanisms, any wasted space may have a high cost.

Similarly, in constructions where a multi-lumen section is slid into the distal section of a single lumen shaft, there are similar but slightly different problems. Again, there are extra steps and processes that worsen the manufacturability and also add a discontinuity in the shaft at the proximal termination of the insert (where kinking can occur). In some cases, in order to reduce the wasted space that occurs by the insert and the space afforded for it, the wall of a single lumen shaft may be thinned out, e.g., to reduce the amount of redundant wall by the insertion of the multi lumen extrusion. In this case, some space is still lost (albeit less) and the performance of the proximal shaft may be compromised and the kink resistance of the distal deflection section may remain suboptimal. For example, two thick-walled sections inside each other are significantly less kink resistant than a single wall thickness of similar or less total wall thickness.

In other state of the art designs, the shaft is left in a more or less optimized state for torque, but the multi-lumen wall may take up much more of the cross section than is ideal, causing a waste of space and also performance limitations (stiffness) in the area of the deflection section. In even yet another design, the multi-lumen extrusion is whittled down in certain areas to remove the outside wall of the peripheral lumens to preserve some space (reduce bulk and stiffness), but the process is even more labor intensive and inconsistent performance wise.

Further problems arise during the incorporation of compression coil stops. EP catheters use compression coils to carry actuator wires to enable deflection without compression loading the proximal portion of the shaft, which otherwise would significantly negatively impact the torque transmission of the shaft through anatomically relevant bends. The compression coils pass through the single lumen portion of the shaft until terminating at the “compression coil stop.” This stop needs to resist the entire maximum deflection force of the distal deflection section, plus friction, plus buffer/safety factor. Such a large force over such a small cross-sectional area means that this stop must be reinforced with more than the typically soft polymers used to construct the catheter. Such stop elements must also be added into either of the above two constructions, which adds to the complexity and to the loss of cross-sectional area, e.g., which must be used for the stop/bond/joint. These constructions provide for new failure modes as well, such as failure of the stop joint.

Yet further problems arise when seeking to incorporate asymmetric deflection. The same complexities listed above are now amplified in creating either two offset/staggered compression coil stops or two offset/staggered pull wire ring locations in order to create different length deflection sections (different length flexible deflection sections are what enable different bend radii/asymmetric deflection in each deflection direction. Both of these methods of incorporating asymmetric deflection are difficult to do from a manufacturing perspective and add further discontinuities to the deflection section.

Further manufacturing and performance problems may arise when electrodes or other sensing elements need to be added to the exterior of the shaft or the deflection section.

Accordingly, there is a need for improved catheters, sheaths, and other tubular devices and methods of their manufacture.

### SUMMARY

The present application is directed to catheters, sheaths, or other tubular devices and to methods for making such devices. More particularly, the present application is directed to catheters, sheaths, or other tubular devices, e.g., steerable tubular devices, including braided or other reinforcement configurations and/or including one or more steering elements for deflecting a distal portion, and/or to methods for making such catheters, sheaths, or other tubular devices. The devices and methods described herein may involve simplified, e.g., single-piece or single-part, construction of tubular bodies for catheter shafts, which may save space and/or provide more robust construction of the resulting devices compared to conventional manufacturing methods.

For example, the methods described herein may provide full EP or other deflectable catheter shaft functionality in a single-piece construction, e.g., with fully continuous braid between sections of the shaft. Optionally, the devices and methods herein may also provide asymmetric deflection functionality, integral compression coil stops, integrated pull-wire shoulders and braid termination, and/or the ability to readily augment the shaft with additional sensing elements. As with shafts from the current state of the art, the single-piece shaft construction may still be augmented with additional components, e.g., a distal tip, electrodes, sensors, balloons, infusion/aspiration channels, optical elements and/or other elements that serve the specific clinical purpose of the finished product while taking advantage of a standardized shaft that is useful across a large range of catheter sizes and uses. Such construction and methods may also enable optimization of the performance, profile, and/or manufacturability of each of the two main sections of a catheter shaft.

In accordance with one example, an apparatus is provided for performing a procedure within a patient's body that includes a tubular member comprising a proximal end, a distal end sized for introduction into a patient's body, a longitudinal axis extending between the proximal end and the distal end, an intermediate portion extending partially between the proximal end and the distal end, and a distal portion extending distally from the intermediate portion at a transition to the distal end; a primary lumen extending between the proximal end and the distal end; an auxiliary lumen extending along the distal portion adjacent the primary lumen from an opening communicating with the primary lumen at the transition to the distal end; a steering element comprising a first portion slidably disposed within the auxiliary lumen and terminating at a first end fixed to the distal end distally beyond the auxiliary lumen and a second portion passing proximally through the opening into the primary lumen and extending to a second end adjacent the proximal end; an actuator on the proximal end coupled to the second end of the steering element such that, actuation of the actuator applies axial tension or compression to the first portion of the steering element, thereby causing the distal portion to bend; and a compression-resistant member disposed around the first portion of the steering element within the primary lumen between the transition and the proximal end for preventing forces from the steering element from transferring to the tubular member proximal to the distal portion.

In accordance with another example, an apparatus is provided for performing a procedure within a patient's body that includes a tubular body comprising a proximal end, a distal end sized for introduction into a patient's body, a longitudinal axis extending between the proximal end and the distal end, an intermediate portion extending partially between the proximal end and the distal end, and a distal portion extending distally from the intermediate portion at a transition to the distal end; a primary lumen extending between the proximal end and the distal end, the primary lumen defining a first segment along the intermediate portion defining a first internal diameter or cross-sectional dimension and a second segment along the distal portion defining a second internal diameter or cross-sectional dimension smaller than the first segment; an auxiliary lumen extending along the distal portion adjacent the primary lumen from an opening at the transition communicating with the primary lumen at the transition to the distal end; a steering clement comprising a first portion slidably disposed within the auxiliary lumen and terminating at a first end fixed to the distal end distally beyond the auxiliary lumen and a second portion passing proximally through the opening into the primary lumen and extending to a second end adjacent the proximal end; an actuator on the proximal end coupled to the second end of the steering element such that, actuation of the actuator applies axial tension or compression to the first portion of the steering element, thereby causing the distal portion to bend; a compression-resistant member disposed around the first portion of the steering element within the primary lumen between the transition and the proximal end for preventing forces from the steering element from transferring to the tubular member proximal to the distal portion; and a plurality of reinforcement members braided continuously along the intermediate and distal portions, wherein, along the distal portion, the plurality of reinforcement members comprising windings wound helically within a wall of the distal portion such that at least some of the windings pass between the primary lumen and the auxiliary lumen and at least some of the windings surround both the primary lumen and the auxiliary lumen.

In accordance with still another example, a single-piece tubular body is provided for a medical comprising that includes a first portion including a first primary lumen segment extending from a first end of the tubular body along a longitudinal axis of the first portion; a second portion adjacent the first portion including a second primary lumen segment communicating with the first primary lumen segment and extending along the longitudinal axis towards a second end of the tubular body, and an auxiliary lumen extending along the second portion adjacent the second primary lumen segment from an opening at a transition between the first and second portions towards the second end; and a plurality of reinforcement members braided continuously along the first and second portions. Optionally, the tubular body may include two or more auxiliary lumens, which may be offset axially relative to one another.

In accordance with still another example, a method is provided for making a tubular body that includes providing a primary mandrel comprising a first section defining a first cross-sectional dimension and a second portion defining a second cross-sectional dimension smaller than the first cross-sectional dimension; braiding reinforcement members around a liner along the first section towards the second section; positioning a secondary mandrel outside the primary mandrel adjacent the second section; braiding the reinforcement members along the second section such that the secondary mandrel is included within a braid of the reinforcement members to define an auxiliary lumen; applying an outer jacket around the primary mandrel after braiding the reinforcement members around the first and second sections; and removing the primary mandrel to define a primary lumen including a first segment corresponding to the first section of the primary mandrel, defining a first portion of the tubular body, and a second segment corresponding to the second section of the primary mandrel, defining a second portion of the tubular body, the auxiliary lumen extending along the second portion adjacent to the second segment.

Other aspects and features of the present invention will become apparent from consideration of the following description taken in conjunction with the accompanying drawings.

### BRIEF DESCRIPTION OF THE DRAWINGS

It is believed the present invention will be better understood from the following description of certain examples taken in conjunction with the accompanying drawings, in which like reference numerals identify the same elements and in which:

FIG. 1A is a perspective view of an example of a catheter, including a primary lumen extending between proximal and distal ends thereof, and including a steerable distal portion.

FIG. 1B is a longitudinal cross-sectional side view of the catheter of FIG. 1A, taken along line 1B-1B showing a steering element extending between the distal end and a handle of the catheter.

FIG. 2A is a cross-sectional side view of the catheter of FIGS. 1A and 1B, taken along line 2A-2A, showing reinforcement members positioned around the primary lumen along an intermediate portion of the catheter.

FIG. 2B is a cross-sectional side view of the catheter of FIG. 1A, taken along line 2B-2B, showing reinforcement members braided around primary and auxiliary lumens of the catheter along the distal portion.

FIG. 3 is a detail of exemplary internal components that may be included in the catheter of FIGS. 1A and 1B showing a transition between an intermediate portion and the distal portion.

FIG. 4 shows an example of a braided assembly that may be used to make the catheter of FIGS. 1A and 1B.

FIGS. 4A-4C are cross-sectional views of the braided assembly shown in FIG. 4 taken along sections 4A-4A, 4B-4B, and 4C-4C, respectively.

FIG. 5A is a schematic of an example of a braiding apparatus for making a reinforced tubular member including multiple mandrels supported by reinforcement members.

FIG. 5B is a front view of an arrangement of horn gears for creating a braided configuration of reinforcement members that may be included in the braiding apparatus of FIG. 5A and including various locations for sources of mandrels.

FIG. 5C is a cross-sectional view of a catheter showing the locations of mandrels corresponding to the different locations for the sources of mandrels shown in FIG. 5B.

FIG. 6 is a side view of an exemplary method for making a tubular body for a catheter or other device including a primary mandrel and secondary mandrels.

FIG. 6A is a cross-section of the mandrel shown in FIG. 6 including a transition with a pair of channels in the primary mandrel for receiving the secondary mandrels.

FIG. 6B is a cross-section of an alternative primary mandrel including a transition with only a single channel for receiving a secondary mandrel.

FIG. 7 is a side view of another example of a single-piece tubular body.

FIGS. 7A-7C are cross-sections of the tubular body shown in FIG. 7 taken along sections 7A-7A, 7B-7B, and 7C-7C, respectively.

FIG. 7D is a cross-section of the tubular body shown in FIG. 7 including a pair of steering elements received in the auxiliary lumens and attached to a pull ring, and compression-resistant members positioned over the steering elements.

FIGS. 8A and 8B are cross-sections of another example of a tubular device including two auxiliary lumens offset axially relative to one another along a steerable portion of a tubular body.

FIGS. 9A and 9B are cross-sections of another example of a tubular device including two auxiliary lumens offset axially relative to one another along a steerable portion of a tubular body.

FIGS. 10A and 1B are cross-sections of another example of a tubular device including two auxiliary lumens offset axially relative to one another along a steerable portion of a tubular body.

FIGS. 11A and 11B are cross-sections of another example of a tubular device including two auxiliary lumens offset axially relative to one another along a steerable portion of a tubular body.

FIG. 12 is a cross-section of an alternative example of a distal portion including stabilization elements embedded adjacent auxiliary lumens.

FIG. 13 is a cross-section of another alternative example of a distal portion including a softer core surrounded by a relatively stiff outer layer.

FIGS. 14A-14C show an example of a pull ring that may be coupled to a pair of steering elements.

The drawings are not intended to be limiting in any way, and it is contemplated that various examples of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

Before the examples are described, it is to be understood that the invention is not limited to particular examples described, as such may, of course, vary. It is also to be understood that the terminology used herein is for the purpose of describing particular examples only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and exemplary methods and materials are now described.

It must be noted that as used herein and in the appended claims, the singular forms “a,” “an,” and “the” include plural referents unless the context clearly dictates otherwise. Thus, for example, reference to “a compound” includes a plurality of such compounds and reference to “the polymer” includes reference to one or more polymers and equivalents thereof known to those skilled in the art, and so forth.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

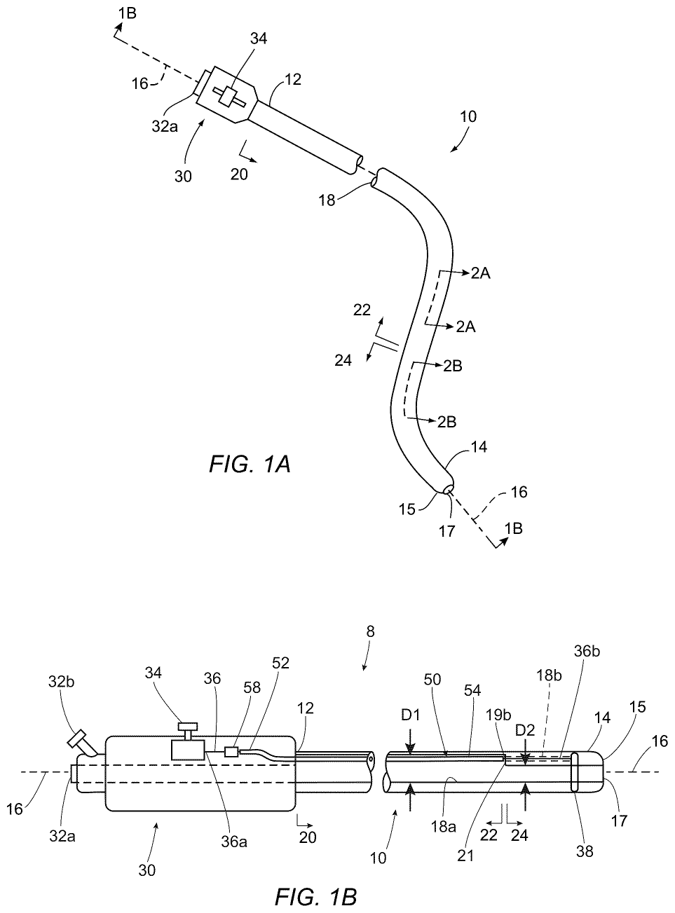

Turning to the drawings, FIGS. 1A and 1B show an example of an apparatus 8 for introduction into a body lumen (not shown), e.g., for performing a diagnostic and/or therapeutic procedure within a patient's body. For example, the apparatus 8 may be a guide catheter, a sheath, a procedure catheter, e.g., an imaging catheter, an ablation and/or mapping catheter, a balloon catheter, or other tubular device sized for introduction into a body lumen, such as a vessel within a patient's vasculature, a passage within a patient's gastrointestinal tract, urogenital tract, reproductive tract, respiratory tract, lymphatic system, and the like (not shown). In various examples, the apparatus 8 may have a length between about ten and one hundred seventy centimeters (10-170 cm), and/or an outer diameter between about four and twenty-four French (4-24 Fr or 1.33-8.0 mm). The outer diameter or non-circular cross-section of the apparatus 8 may be substantially uniform or variable along the length of the apparatus 8.

Generally, the apparatus 8 includes an clongate tubular body or member 10

including a proximal end 12, a distal end 14 sized for insertion into a body lumen, a longitudinal axis 16 extending between the proximal and distal ends 12, 14, and one or more lumens 18 extending at least partially between the proximal and distal ends 12, 14. For example, as best seen in FIG. 1B, the apparatus 10 may include a central or primary lumen 18a extending from the proximal end 12 to an outlet 17 in the distal end 14. The primary lumen 18a may be sized for receiving or carrying one or more instruments or devices, e.g., a guide wire, procedure catheter, balloon catheter, ablation catheter, cardiac lead, needle, or other instrument (not shown), one or more wires or other conductors, one or more optical fibers, one or more tubes or accessory lumens, one or more mechanical elements, one or more sensors, and/or sized for delivering and/or removing fluids or other flowable agents or materials therethrough.

In the example shown in FIGS. 1A and 1B, the primary lumen 18a may exit at or communicate with the outlet 17 in a distal tip 15 of the distal end 14, e.g., to allow a guidewire or other instrument (not shown) to pass therethrough and/or for delivering or aspirating fluid therethrough. Alternatively, the primary lumen 18a may be enclosed, i.e., without the outlet, e.g., terminating within or adjacent the distal end 14, e.g., by an electrode, cap, or other component (not shown) to isolate the primary lumen 18a and/or elements carried therein from the environment outside the apparatus 8.

In addition to the primary lumen 18a, the tubular body 10 includes one or more auxiliary lumens 18b, e.g., extending at least partially between the proximal and distal ends 12, 14 adjacent the primary lumen 18a. For example, as shown in FIG. 1B, a single auxiliary lumen 18b may be provided that extends along the distal portion 24. The auxiliary lumen(s) 18b may be sized for slidably receiving a pull wire or other steering element 36 therein, e.g., to bend or otherwise deflect the distal portion 24, as described further elsewhere herein. As shown, the auxiliary lumen 18b is generally radially offset from the longitudinal axis 16 along the length of the distal portion 24 and extends substantially parallel to the longitudinal axis 16. Alternatively, the radial and/or circumferential position of the auxiliary lumen(s) 18b may change relative to the primary lumen 18a and/or other components of the tubular body 10 at various locations along the length of the apparatus 8, if desired.

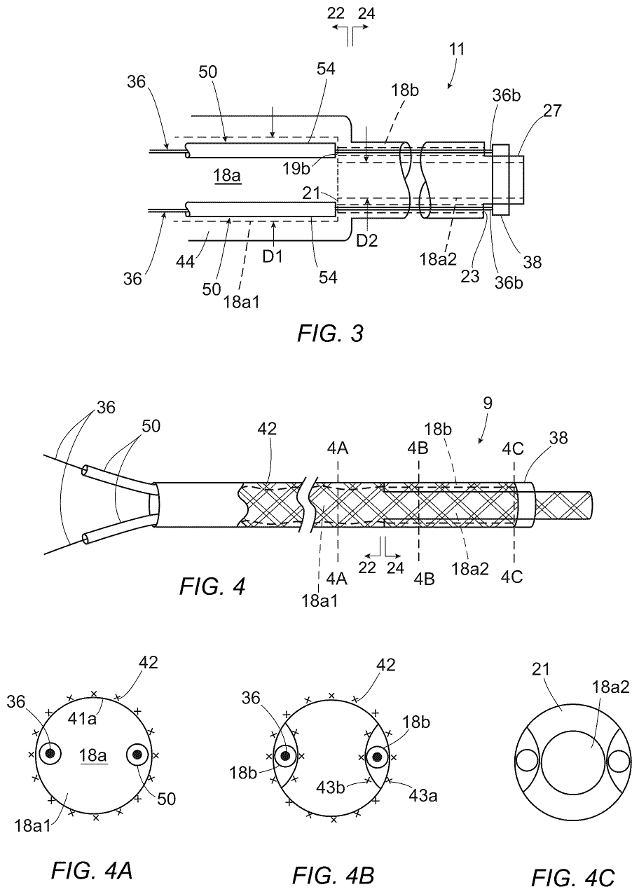

Alternatively, as shown in FIG. 3, a pair of auxiliary lumens 18b may be

provided along the distal portion 24 of the tubular body 10. As shown, the auxiliary lumens 18b may be provided on opposite sides of the tubular body 10, e.g., offset about one hundred eight degrees (180°) from one another around the circumference of the tubular body 10. Optionally, if desired, the tubular body 10 may include one or more additional lumens (not shown), e.g., one or more additional steering element lumens, conductor lumens, inflation lumens (e.g., if the apparatus 8 includes one or more balloons, not shown on the distal end 14), and/or accessory lumens, e.g., within a wall of the tubular body 10. For example, in some applications, it may be desirable to provide one or more lumens offset circumferentially between the auxiliary lumens 18b, e.g., a lumen offset about ninety degrees (90°) from the auxiliary lumens 18b, which may receive one or more cables, wires, or provide inflation lumens.

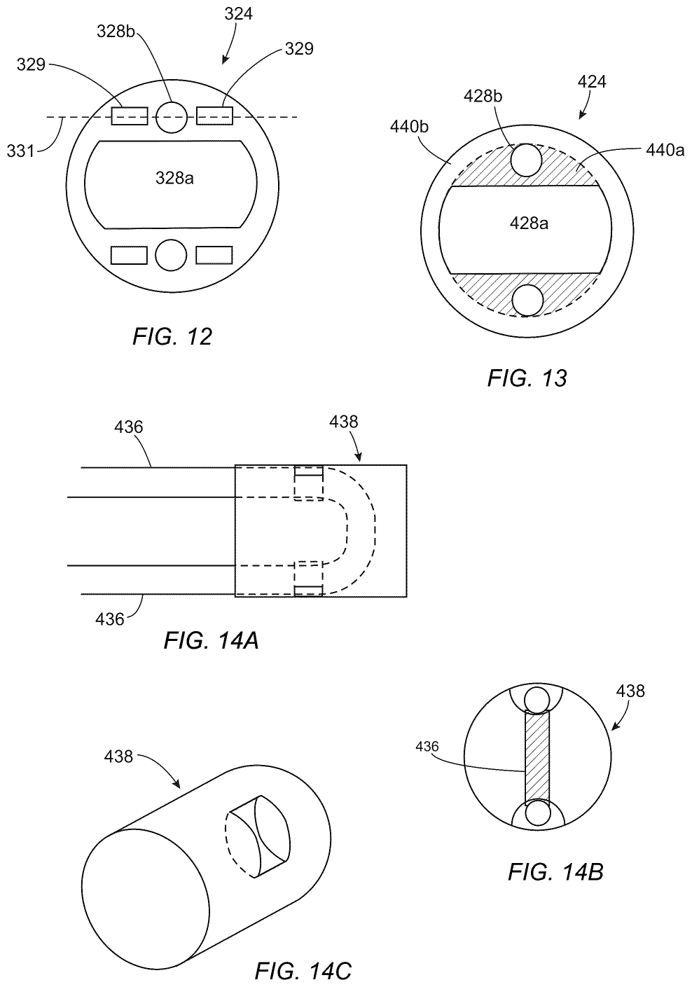

Optionally, one or more stabilization elements may be provided adjacent the auxiliary lumen(s). For example, as shown in FIG. 12, a pair of hinges or other relatively flat stabilization members 329 may be embedded in a distal portion 324 of a tubular body, e.g., on either side of each auxiliary lumen 318b, e.g., aligned along a plane 331. The stabilization member(s) 329 may enhance desired bending of the distal portion 324 when a steering element (not shown) with the auxiliary lumen 318b is actuated, e.g., within a plane orthogonal to the plane 331 of the stabilization members 329. In addition or alternatively, as shown in FIG. 13, the material of a steerable portion including one or more auxiliary lumens 428b adjacent a primary lumen 428a may be constructed to provide a desired bending profile. For example, as shown, a relatively softer core 440a may be provided, e.g., having a Durometer of about 35 D, and a relatively stiff outer layer 440b may be provided, e.g., having a Durometer of about 55 D, which may reduce overall stiffness of the steerable portion and/or mitigate unpredictable deflection.

Optionally, the primary and/or auxiliary lumen(s) may have a variety of cross-sectional shapes and/or sizes, e.g., a substantially circular shape, an elliptical or oval shape, a substantially rectangular shape, a triangular shape, a pair of overlapping circles shape, and the like, e.g., as described elsewhere herein and similar to the devices disclosed in U.S. Publication No. 2014/0323964, the entire disclosure of which is expressly incorporated by reference herein. The shape and/or size of the primary and/or auxiliary lumen(s) may be substantially uniform along their lengths or may vary at different locations, as described elsewhere herein.

For example, as shown in FIGS. 1B and 3, the primary lumen 18a may have a first segment 18a1 defining a first diameter or cross-section DI along at least the intermediate portion 22 and a second segment 18a2 defining a second diameter or cross-section D2 along the distal portion 24 that is smaller than the first diameter D1, as described further elsewhere herein.

In this example, the primary lumen 18a may include an end wall 21 providing a transition between the first and second segments 18a1, 18a2 and, therefore, between the intermediate portion 22 and the distal portion 24. Also in this example, the auxiliary lumen(s) 18b may communicate with an opening 19b in the end wall 21, which may allow the steering element 36 to pass between the primary lumen 18a and the auxiliary lumen 18b, as described further elsewhere herein. The end wall 21 may be substantially flat, e.g., substantially perpendicular to the longitudinal axis 16 as shown. Alternatively, the end wall may have a tapered or other shape (not shown) transitioning between the first and second segments 18a1, 18a2.

Returning to FIG. 1A, the distal end 14 may include a tapered, rounded, or otherwise shaped distal tip 15, e.g., to provide a substantially atraumatic tip and/or to facilitate advancement or navigation through various anatomy. In addition or alternatively, the distal end 14 may include one or more therapeutic and/or diagnostic elements, e.g., one or more balloons, stents, sensors, electrodes, ablation elements, thermocouples, steering mechanisms, imaging devices, helical anchors, needles, and the like (not shown), depending upon the particular intended application for the apparatus 8. Such elements may be provided on the distal portion 24 or on an extension beyond the distal portion 24, as desired. Further, in addition or alternatively, the distal end 14 and/or distal portion 24 may include one or more markers or other features to enhance radiopacity and/or visibility under ultrasound, MRI, or other imaging modalities, e.g., by mounting one or more platinum elements on the distal end 14, doping one or more regions of the distal end 14 with tungsten or barium sulfate, and/or other methods known in the art.

With continued reference to FIGS. 1A and 1B, the proximal end 12 may include a handle or hub 30, e.g., configured and/or sized for holding and/or manipulating the apparatus 8 from the proximal end 12. In addition, the handle 30 may include one or more ports, e.g., a port 32a communicating with the primary lumen 18a, or other respective lumens (not shown). Optionally, the port 32a may include one or more valves, e.g., a hemostatic valve (also not shown), which may provide a substantially fluid-tight seal, while accommodating insertion of one or more instruments or fluids into the primary lumen 18a. Optionally, as shown in FIG. 1B, a side port 32b may be provided on the handle 30, e.g., for delivering fluid into and/or aspirating fluid from the primary lumen 18a, e.g., around an instrument inserted into the primary lumen 18a through the first port 32a. Optionally, the handle 30 and/or proximal end 12 may include one or more connectors, such as luer lock connectors, electrical connectors or cables, and the like, for connecting other devices (not shown) to the apparatus 10, such as syringes, displays, controllers, and the like (also not shown).

In addition, the handle 30 may include one or more actuators, such as sliders,

buttons, switches, rotational actuators, and the like, e.g., for activating and/or manipulating components (also not shown) on the distal end 14 or otherwise operating the apparatus 8. For example, as shown in FIGS. 1A and 1B, an actuator 34 may be provided that is coupled to a proximal end 36a of the steering clement 36 within the auxiliary lumen 18b. The actuator 34 may be movable, e.g., slidable axially, rotated around the axis 16, and the like, to apply axial, e.g., proximal, tension to the steering element 36, e.g., to cause the distal portion 24 to deflect, as described elsewhere herein. In a device that includes two opposite steering elements, e.g., similar to the example shown in FIG. 3, the handle may include an actuator (not shown) coupled to both steering elements 36 to apply/release tension on the respective steering elements when the actuator is moved in first and second directions, or separate actuators (also not shown) may be coupled to the respective steering elements 36 such that the steering elements may be actuated independently of one another, if desired.

The steering element(s) 36 may be formed from materials capable of substantially transferring any axial forces applied at the proximal end to the distal end 14, as is known in the art, e.g., a wire or other solid or hollow elongate member that is substantially incompressible or extendable axially yet flexible to accommodate bending of the tubular body 10. Alternatively, the steering element 36 may be formed from multiple strands, e.g., such as a braided or stranded cable or thread, which may provide a rifled outer surface, which may reduce friction. Optionally, the steering element(s) 36 may include a coating, e.g., PTFE, parylene, silicone, or other lubricious material, an outer sleeve, e.g., formed from HDPE, PTFE, and the like, to reduce friction between the steering element and the wall of the auxiliary lumen 18b. Alternatively or in addition, the inner surface of the auxiliary lumen 18b may be formed from lubricious material and/or may include one or more coatings, as described elsewhere herein.

In addition, the apparatus 8 may include one or more additional components that interact with the steering element(s) 36 to control deflection of the distal portion 24. For example, as shown in FIG. 1B, a pull ring or other anchor 38 may be provided adjacent the distal end 14 of the tubular body 10, e.g., fixed to the distal end 14, to which a distal end 36b of the steering element 36 may be attached to translate axial tension from the steering element 36 to the distal portion 24. In addition, a compression-resistant member 50 maybe provided that surrounds or is otherwise positioned adjacent to at least a portion of the steering element 36, e.g., to prevent forces from the steering element 36 from transferring to the tubular body 10 proximal to the distal portion 24, as described elsewhere herein. Optionally, one or more stops may be provided that may limit axial movement of the compression-resistant member 50, e.g., a stop 58 within the handle 50 adjacent a proximal end 52 of the compression-resistant member 50 and/or adjacent the end wall 21 and a distal end 54 of the compression-resistant member 50, also as described further elsewhere herein.

During use, the actuator 34 may be activated, e.g., directed proximally or distally relative to the handle 50 and/or the proximal end 12, to apply an axial force to the steering element 36. For example, the actuator 34 may be directed in a first direction, e.g., proximally, to apply tension when the steering clement 36 is pulled, and in a second direction, e.g., distally, to release tension and/or apply compression when the steering element 36 is advanced. Because the steering element 36 is slidable within the auxiliary lumen 18b, the axial force is translated and applied to the distal end 36b coupled to the pull ring 38. Further, because the auxiliary lumen 18b is offset from the longitudinal axis 16 along at least the distal portion 24, the axial force applies a bending moment, thereby causing the distal portion 24 to curve or otherwise bend in a desired plane or other manner.

In the example shown in FIG. 1B, the compression-resistant member 50 surrounds a portion of the steering clement 36, e.g., extending within the primary lumen 18a from the proximal end 12 of the tubular body 10 through the intermediate portion 22 and terminating before the distal portion 24, to prevent forces from the steering element 36 from transferring to the tubular body 10 along the intermediate portion 22 and/or proximal to the distal portion 24. For example, the compression-resistant member 50 may be sized to slidably receive the steering element 36 therethrough, e.g., thereby preventing bending forces from the steering clement 36 being transferred to the tubular member material along portion(s) where the compression-resistant member 50 is provided, e.g., along the intermediate portion 22.

In one example, the compression-resistant member 50 may be a tightly-wound coil, e.g., with coils abutting one another in a relaxed or low potential energy state, such that the coil cannot be compressed axially. Alternatively, the coil 50 may be formed from metal, steel, polymers, or composite materials, e.g., one or more stainless steel or Nitinol wires having a round or rectangular cross-section. Alternatively, other tubular or cylindrical structures may be provided for the compression-resistant member 50, which may slidably receive the steering clement 36 therethrough. For example, the compression-resistant member 50 may include a counter-wound coil tube, a coil tube with one or more attached tensile elements, a laser cut tube, a densely braided polymer tube, and the like (all not shown).

The compression-resistant member 50 may be free floating or otherwise slidably received within the primary lumen 18a, e.g., with proximal and distal ends 52, 54 thereof remaining uncoupled to the tubular body 10. Alternatively, one or both ends may be fixed or stopped relative to the tubular body 10. For example, as shown in FIG. 1B, a stop 58 may be provided within the handle 50 that is axially fixed relative to the handle 50 and the proximal end 52 of the compression resistant member 50 may be positioned over the steering element 36 immediately distal to the stop 58 (without being mechanically coupled to the stop 58) to prevent proximal movement of the proximal end 52. Similarly, the distal end 54 of the compression-resistant member 50 may be positioned immediately adjacent the end wall 21 within the primary lumen 18a (without being mechanically coupled to the end wall 21) to prevent distal movement of the distal end 54. Optionally, a stop or other reinforcement (not shown) may be provided on or in the end wall 21 to further support the distal end 54, e.g., to prevent the distal end 54 from penetrating into the material of the end wall 21.

Optionally, the compression-resistant member 50 may be subjected to pre-compression, e.g., between the proximal stop 58 and the end wall 21. For example, during assembly, the stop 58 may be initially positioned within the handle 50 such that the opposite ends 52, 54 of the compression-resistant member 50 contact the stop 58 and end wall 21. The stop 58 may then be adjusted, e.g., directed distally before being secured relative to the handle 50, thereby creating an axial compression in the compression-resistant member 50 between the opposite ends 52, 54. Such pre-compression may ensure that the compression-resistant member 50 remains under axial compression for the life of the resulting apparatus 8, e.g., if the material of the tubular body 10 softens or stretches slightly after final assembly.

Alternatively, the distal end 54 may be embedding within, attached, or otherwise fixed relative to the end wall 11, e.g., by one or more of bonding with adhesive, heat welding, sonic welding, incorporation of a secondary stop element, and the like. In one example, the compression-resistant member 50 may be fixed at its distal end 54 with an additional stop for reinforcement whereas the proximal end 52 may be left free to slide over the steering element 36 adjacent the proximal stop 58, e.g., such that path length changes due to manipulation of the catheter may be accommodated without transferring these forces to the tubular body 10.

The diameter or other cross-section of the auxiliary lumen 18b may be smaller than an outer diameter or other cross-section of the distal end 54 of the compression-resistant member 50 to prevent the distal end 54 from entering the auxiliary lumen 17b. In one example, the auxiliary lumen 18b may have a diameter of about 0.012 inch (0.30 mm) and the distal end 54 may have an outer diameter of about 0.020 inch (0.50 mm). Optionally, a radial dimension of the end wall 21 (e.g., width transverse to the longitudinal axis 16) may be smaller than the diameter of the distal end 54 of the compression-resistant member 50. Consequently, the distal end 54 may only partially contact the end wall 21. The steering element 36 within the compression-resistant member 50 and auxiliary lumen 18b may prevent lateral movement of the distal end 54, thereby preventing distal movement of the distal end 54 of the compression-resistant member 50.

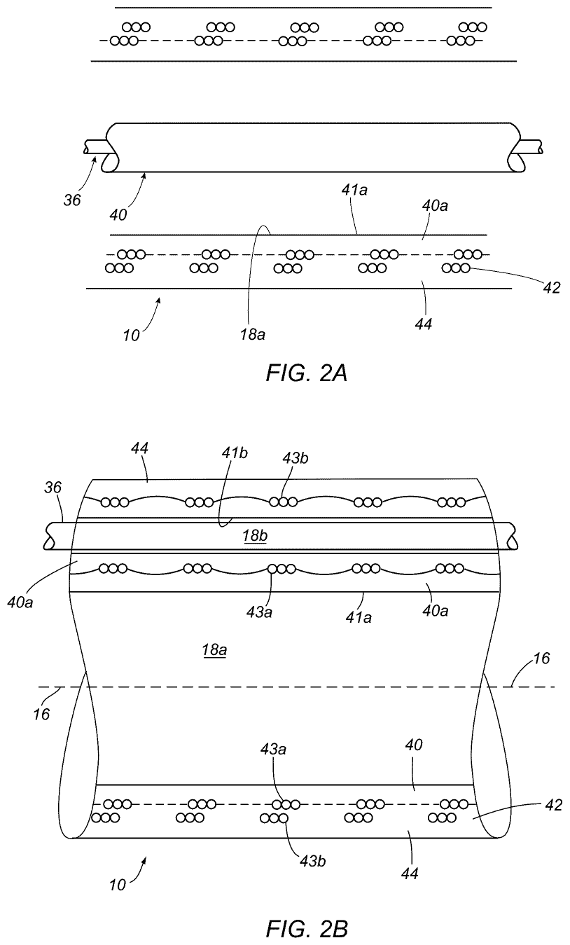

Generally, as shown in FIGS. 2A and 2B, the tubular body 10 may be constructed to include an inner liner 40, e.g., at least partially or entirely surrounding or otherwise defining the primary lumen 18a, a reinforcement layer 42 surrounding the inner liner 40, and an outer jacket 44 surrounding and/or encasing the reinforcement layer 42, each of which may extend at least partially between the proximal and distal ends 12, 14 of the apparatus 10. The reinforcement layer 42 and/or outer jacket 44 may be permanently attached to the inner liner 40, e.g., by one or more of laminating, adhering, adhesive bonding, ultrasonic welding, reflowing or other heating, and the like.

In one example, as shown in FIG. 2A, the primary lumen 18a may include an inner liner 40a defining an inner surface 41a thereof. The inner liner 40a may be formed from lubricious material, e.g., PTFE or a fluoropolymer, to provide a lubricious inner surface 41a. Alternatively, the inner liner 40 may be formed from one or more layers of thermoplastic or other polymeric material including one or more coatings on the inner surface 41a having desired properties, e.g., a hydrophilic and/or lubricious coating, e.g., similar to the liners disclosed in U.S. Pat. Nos. 7,550,053 and 7,553,387, and U.S. Publication No. 2009/0126862, the disclosures of which are expressly incorporated by reference herein. In a further alternative, the inner surface 41a may be coated with a hydrophobic coating free of per-and polyfluoroalkyl substances (PFAS).

Optionally, as shown in FIG. 2B, an inner liner 40b may also at least partially surround the auxiliary lumen 18b, which may be formed from a lubricious material and/or may include one or more coatings on its inner surface 41b, similar to the inner liner 40a. The inner surface 41b of the auxiliary lumen 18b may have a substantially uniform cross-section, as shown in FIG. 2B. Alternatively, the inner surface 41b of the auxiliary lumen 18b may have a textured or other variable cross-section along, e.g., along its length and/or about its circumference (not shown).

Optionally, any or all of the inner liner 40a, reinforcement layer 42, and/or outer jacket 44 may be formed from multiple layers of like or different materials (not shown), e.g., to provide desired material properties in the different portions of the apparatus 10. In one example, the outer jacket 44 may be formed from PEBAX, nylon, urethane, and/or other thermoplastic material, e.g., such that the material of the outer jacket 44 may be heated and reflowed and/or otherwise formed around the components defining the lumens 18, e.g., as described elsewhere herein.

One or more of the layers of the tubular body 10 may have a substantially homogenous construction between the proximal and distal ends 12, 14. For example, the reinforcement layer 42 may be applied substantially continuously between the proximal and distal portions 20-24 of the tubular body 10. As explained elsewhere herein, such single-piece construction may provide several advantages over attaching separate tubular components together to provide the tubular body 10, e.g., avoiding discontinuities, connection points that may compromise device performance, and the like. In addition, if desired, the liner 41a may extend substantially continuously and uniformly between the proximal portions 20-24.

Alternatively, the construction may vary along the length of the tubular body 10 to provide desired properties, e.g., between proximal, intermediate, and distal portions 20, 22, 24. For example, the proximal portion 20 of the tubular body 10 adjacent the proximal end 12 may be substantially rigid or semi-rigid, e.g., providing sufficient column strength to allow the distal end 14 of the tubular body 10 to be pushed or otherwise manipulated from the proximal end 12, while the distal portion 24 may be substantially flexible to accommodate bending and/or introduction into tortuous anatomy. As described elsewhere herein, the distal portion 24 of the tubular body 10 may be steerable, i.e., may be bent, curved, or otherwise deflected substantially within a steering plane.

With continued reference to FIGS. 2A and 2B, the reinforcement layer 42 may include one or more reinforcing members 43, e.g., wound in a braided or other helical configuration around the inner liner 40a, e.g., using a braiding apparatus such as that described in U.S. Pat. Nos. 9,427,551, 10,071,222, and 10,065,015, the entire disclosures of which are expressly incorporated by reference herein. The outer jacket 44 may include one or more tubular layers surrounding the reinforcement layer 42 and/or between the reinforcement layer 42 and the inner liner 40a. In one example, the reinforcement layer 42 may include one or more, or a plurality of, round or flat (e.g., rectangular, elliptical, or flat oval) wires, filaments, strands, or other reinforcement members 43, e.g., formed from metal, such as stainless steel, plastic, such as PEEK, glass, woven or twisted fibers, such as aramid, and the like, or composite materials.

For example, a plurality of reinforcement members 43 may be braided around the inner liner 40a, e.g., with each reinforcement member 43 having the same material and/or shape. Alternatively, the reinforcement members 43 may have different sizes, materials, and/or shapes, e.g., a first size or shape extending helically in a first direction and a second size or shape (different than the first) extending helically in a second direction (e.g., opposite the first direction).

The reinforcement layer 42 may be configured to substantially transfer torsional forces between the proximal and distal ends 12, 14, e.g., to allow the apparatus 10 to be twisted from the proximal end 12 to rotate the distal end 14 about the longitudinal axis 16 within a patient's body. In addition, the reinforcement layer 42 may allow the distal end 14 of the apparatus 10 to be advanced or otherwise manipulated within a patient's body from the proximal end 12 without substantial risk of buckling and/or kinking. Optionally, if desired the pitch of the reinforcement layer 42 may be varied along the length of the apparatus 10, e.g., in order to optimize mechanical properties of various segments or portions of the apparatus 10.

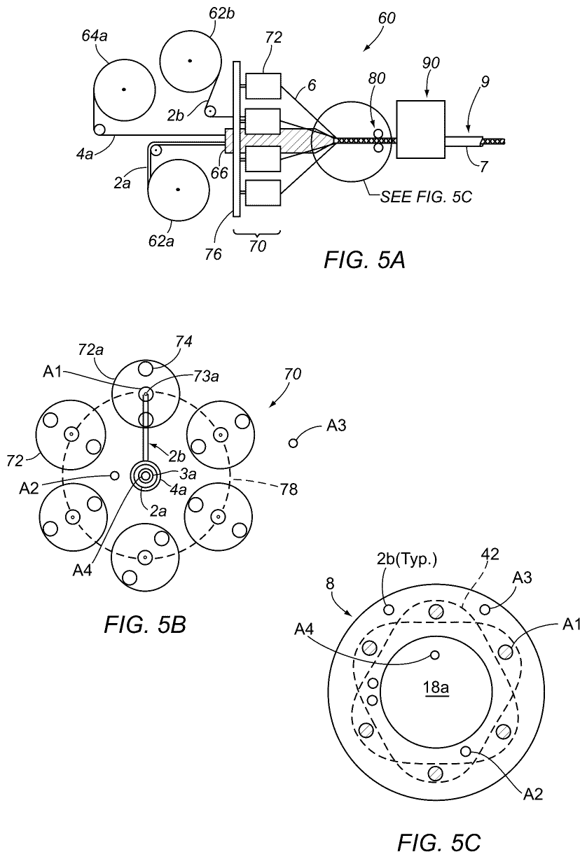

In addition, the location of the reinforcement layer 42 may vary relative to the primary lumen 18a and/or auxiliary lumen(s) 18b, e.g., if the auxiliary lumen 18b transitions to different radial locations within the wall of the tubular body 10. For example, as shown in FIGS. 2A and 3, along the intermediate portion 22 of the tubular body 10, reinforcement members 43 may be braided substantially continuously around the liner 40a, e.g. applied over a mandrel using a braiding apparatus, such as the apparatus 60 shown in FIG. 5A, and then the outer layer 44 may be applied to provide a finished outer surface of the tubular body 10. For example, FIG. 4 shows an example of a braided assembly that may be created using a braiding apparatus and then incorporated into the tubular body 10 and/or apparatus 8, e.g., after applying an outer layer 44 around the braided assembly as described in the patents incorporated by reference elsewhere herein.

As a result, when the tubular body 9 is fully assembled, along the intermediate portion 22, the steering element 36 and compression-resistant member 50 may be located loose within the primary lumen 18a surrounded by the layers 40-44 of the tubular body 10, e.g., as shown in FIGS. 1B, 2A, and 4A. Within the distal portion 24, the steering element 36 may enter the opening 19b and extend along the auxiliary lumen 18b.

As shown in FIGS. 2B and 4B, along the distal portion 24, the auxiliary lumen 18b may extend through the braid of the reinforcement layer 42, e.g., such that some reinforcement members 43a pass between the auxiliary lumen 18b and the primary lumen 18a and some reinforcement members 43b surround both the auxiliary lumen 18b and the primary lumen 18a, e.g., similar to the apparatus and methods described in the patents incorporated by reference elsewhere herein. Such construction may minimize risk of the steering element 36 tearing through the wall of the tubular body 10 along the distal portion 24 when tension is applied to deflect the distal portion 24. Alternatively, in some applications, it may be desirable to position the auxiliary lumen 18b entirely inside the reinforcement layer or entirely outside the reinforcement layer along the distal portion 24.

Optionally, the auxiliary lumen 18b may be further reinforced, e.g., without substantially impacting the bendability of the distal portion 24. For example, a coil (not shown) may be provided that surrounds the auxiliary lumen 18b, e.g., embedded within the material of the distal portion 24. Such a coil may be “open wound,” i.e., have a pitch that is greater than a diameter of the coil wire, such that the coil will accommodate bending of the distal portion 24. In addition or alternatively, additional reinforcement members (not shown) may be braided around the auxiliary lumen and/or a strong liner material may be used around the auxiliary lumen.

Further, as shown in FIGS. 3 and 4C, at a distal end of the distal portion 24, e.g., adjacent the distal tip 15, the steering element 36 may pass outside the reinforcement layer 42, e.g., such that reinforcement members 43 only surround the smaller diameter primary lumen segment 18a2. This transition may facilitate attaching the distal end 36b of the steering element 36 to the pull ring 38, which may be positioned over the reinforcement layer 42 adjacent the distal end 14 during assembly, e.g., as shown in FIG. 4. Alternatively, the steering element 36 may remain within the reinforcement members beyond the distal portion. In this alternative, the distal end of the steering element may be connected to a pull ring or other tip that fits into the extension beyond the distal portion (not shown).

Various methods may be used for manufacturing and/or assembling any of the devices described herein. For example, FIGS. 5A and 5B show an example of an apparatus 60 for making one or more tubular bodies, such as catheters and/or components for catheters, sheaths, or other tubular devices 8. Generally, the apparatus 60 includes a plurality of sources 62, 64 of mandrels 2 and/or liners 4, a guide 66, a source 70 of reinforcement members 6, a drive mechanism 80, and, optionally, a source 90 of jacket material 7.

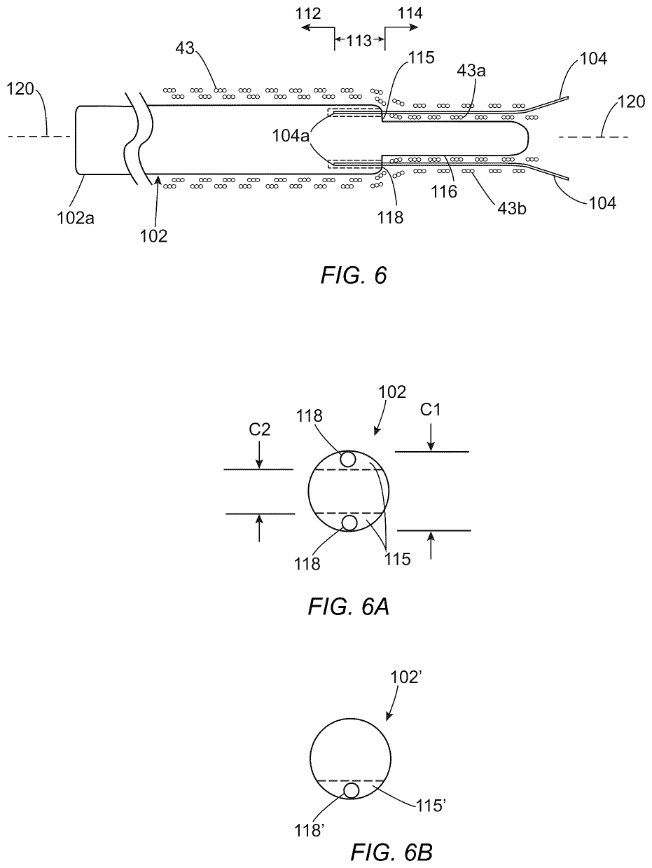

While mandrels, liners, and/or jackets may be provided in discrete segments (not shown), the apparatus 50 may allow for substantially continuous fabrication of tubular bodies, e.g., wrapping liner material 4a around a primary mandrel 2a (or the primary mandrel 2a may include a tubular or other liner material provided around it on the source 62, e.g., similar to the liners disclosed in the references incorporated by reference elsewhere herein). Optionally, the primary mandrel 2a may include a variable cross-section and/or features to facilitate making a single-piece tubular body 10 for the apparatus 8, e.g., to create a braided assembly such as that shown in FIG. 4. For example, as shown in FIG. 6, the primary mandrel may define a length corresponding to a desired braided assembly with one or more channels, e.g., an enclosed lumen, an open groove, and the like provided at desired locations along the mandrel to receive one or more secondary mandrels and/or steering elements. In addition, the primary mandrel may include one or more step-downs or changes in size and/or shape, as described elsewhere herein.

As used herein, “substantially continuous” means that the apparatus 60 and/or method may operate indefinitely, i.e., to make as few as one or as many as hundreds or thousands of tubular bodies 9, e.g., by substantially simultaneously feeding components of the tubular bodies 9 from sources 62, such as reels, through components of the apparatus 60 until the sources 62 are depleted, whereupon new source(s) may be loaded onto the apparatus 60 and the process continued. Alternatively, the apparatus 60 may be used to create discrete lengths of single-piece or single-part tubular bodies, e.g., if the mandrels and/or liners are provided in specific lengths corresponding to one or more individual tubular devices (not shown). In a further alternative, some of the operations may be performed substantially continuously, while other operations are performed on components intended for one or more individual tubular devices.

With particular reference to FIG. 5A, the apparatus 60 may include one or more sources 62 of mandrels 2 and, optionally, one or more sources 64 of liner material 4, which may be fed into a guide 66 to define lumens of the tubular bodies 9. For example, a first reel 62a may include an clongate primary mandrel 2a, e.g., shaped and/or configured to define a primary lumen (not shown) of the tubular bodies 9. Similarly, a second reel 62b may include an elongate auxiliary or secondary mandrel 2b, e.g., shaped and/or configured to define a secondary or auxiliary lumen (also not shown) of the tubular bodies 9. As described further below, the second reel 62b or other source of secondary mandrel may be located at one of a plurality of available locations during operation to configure the tubular bodies 9 in a desired manner. Optionally, if additional lumens are desired for the tubular bodies 9, one or more additional secondary mandrels may be provided (not shown), which may also be moved to one or more locations. The mandrels 2 may have desired cross-sectional shapes and/or sizes

corresponding to the desired cross-sections of the lumens, e.g., substantially circular or other shapes, as described elsewhere herein. The mandrels 2 may be a solid or hollow wire or other cylindrical member having a diameter (or other cross-section) corresponding to the diameter of the lumen to be lined by the liner material 4a, e.g., between about 0.005-0.300 inch (0.125-7.5 mm), 0.014-0.092 inch (0.35-2.3 mm), or 0.014-0.045 inch (0.35-1.15 mm). In various examples, the mandrels 2 may be formed from beading or monofilament material, for example, lubricious material, e.g., PTFE or other fluoropolymer, silicone-treated Acetal, PTFE-coated stainless steel, Parylene-coated stainless steel, silver coated copper, and the like, having sufficient flexibility to allow the mandrels 2 to be wound onto a source reel 62 and/or onto a take-up reel (not shown) after being incorporated into a tubular body 9.

Optionally, a source 64 of liner material 4 may be provided for one or both mandrels 2. For example, as shown, a source 64a of liner material 4a is provided such that the liner material 4a may be wrapped at least partially around the primary mandrel 2a, e.g., as the primary mandrel 2a and liner material 4a are fed through the guide 66. The liner material 2a may be formed from lubricious material and/or may include one or more coatings (not shown) on an inner surface thereof oriented towards the primary mandrel 2a, which may provide an inner liner for a primary lumen of the resulting tubular bodies 9.

For example, the liner material may include a base material, e.g., a relatively thin-walled polymer sheet having a width corresponding to the circumference of the corresponding mandrel, e.g., thermoplastics, such as polyether block amide, urethane, nylon, and the like, fluoropolymers, such as PTFE, FEP, TFE, and the like, thermoset, and thermoform plastics, such as polyimide or polyester, and the like. In various examples, the liner material may have a thickness between about 0.0001-0.050 inch (0.0025-1.25 mm), 0.0001-0.003 inch (0.0025-0.076 mm), 0.0001-0.0015 inch (0.0025-0.038 mm), or 0.0005-0.002 inch (0.0125-0.05 mm).

Optionally, if desired a source of liner material may also be provided for the secondary mandrel 2b and/or for other secondary mandrels (not shown for simplicity). In this option, a guide (not shown) may be provided for wrapping the liner material around the secondary mandrel 2b, e.g., before the secondary mandrel 2b is positioned adjacent the primary mandrel 2a. In an alternative embodiment, tubular liner material may be provided on one or both mandrels when loaded on the source 52, and/or may be fed onto the desired mandrel in discrete segments (not shown) before passing the mandrels 2 through the guide 60 or horn gear 72.

With additional reference to FIGS. 5A and 5B, the source 70 of reinforcement members 6 may provide one or more, e.g., a plurality of, reinforcement members 6 that may be wrapped around the mandrels 2, e.g., upon exiting the guide 66. In the example shown in FIG. 5B, the reinforcement source 70 may include an arrangement of horn gears 72, e.g., mounted in a generally circular configuration around the guide 66, for example, to a base or other support structure 76. The horn gears 72 may be free to rotate about their individual central axes but may be substantially fixed translationally relative to one another and the guide 66. The horn gears 72 may pass one or more carriers 74 of reinforcement members 6 around the path 78, e.g., in a clockwise and/or counterclockwise direction, e.g., with at least some of the carriers travelling clockwise and some travelling counterclockwise, e.g., to create a braided pattern. The carriers 74 may be loaded onto the horn gears to create a variety of patterns, e.g., one-over-one-under (diamond pattern), two-over-two-under (herring bone pattern), one-over-one-under with two reinforcement members running side by side (tow), and/or other patterns, as are known in the art.

In addition, the secondary mandrel 2b may be moved to different locations relative to the horn gears 72, e.g., to reposition the secondary mandrel 2b relative to the primary mandrel 2a and/or reinforcement members 6. For example, as shown in FIG. 5B, during operation of the apparatus 60, the source of secondary mandrel 2b may be positioned at locations A1, A2, A3, or A4, e.g., for a predetermined time and/or distance along the primary mandrel 2a, and, as desired, moved to one of the other locations one or more times. Thus, in this manner, the location of the secondary mandrel 2b may be adjusted, which may result in the location of an auxiliary lumen defined by the secondary mandrel 2b being moved to desired locations and/or omitted, e.g., as shown in FIG. 5C and described further elsewhere herein.

For example, in position A1 shown in FIG. 5A, one of the horn gears 72a may include a passage 73a therethrough, e.g., aligned with the central axis of the horn gear 72a, and the secondary mandrel 2b may pass through the passage 73a, e.g., from the source 52b towards the primary mandrel 2a where it exits the guide 60. If liner material is wrapped or otherwise disposed around the secondary mandrel 2b, a guide (not shown) may be provided before, after, or within the passage 73a to wrap or otherwise dispose the liner material around the secondary mandrel 2b. Optionally, if additional secondary lumens are to be provided in the tubular bodies 9, one or more additional horn gears may also include such passage(s) and/or guide(s) for guiding corresponding secondary mandrel(s) therethrough.

As described further below, in this location, the secondary mandrel 2b may be at least partially braided into the reinforcement members 6 adjacent the primary mandrel 2a, i.e., with some reinforcement members 6 surrounding both the primary mandrel 2a and the secondary mandrel 2b, and some reinforcement members 6 surrounding only the primary mandrel 2a, as identified by secondary mandrels A1 shown in FIG. 5C. For example, in the A1 position, an auxiliary lumen 18b may be created along the distal portion 24, e.g., for the tubular body 10 shown in FIGS. 1A, 1B, or two auxiliary lumens 18b may be created, e.g., for the tubular body 11 shown in FIG. 3. As described further elsewhere herein, the secondary mandrel 2b may provide a steering element for each auxiliary lumen 18b or, alternatively, during assembly, the secondary mandrel 2b may be removed and replaced by a steering element inserted into the auxiliary lumen 18b.

By comparison, in location A2, i.e., with the secondary mandrel 2b directed immediately adjacent the primary mandrel 2a, e.g., through the guide 66, all of the reinforcement members 6 may surround both the primary mandrel 2a and the secondary mandrel 2b, thereby positioning the secondary mandrel 2b closest to the primary mandrel 2a along the tubular device 8. In location A3, i.e., with the secondary mandrel 2b outside the path of the horn gears 72, e.g., outside the path 78 shown in FIG. 5B, or otherwise directed towards the primary mandrel 2a after the braiding operation, all of the reinforcement members 6 may only surround the primary mandrel 2a and the secondary mandrel 2b may remain outside all of the reinforcement members 6, e.g., closest to the outer surface of the tubular device 8 shown in FIG. 5C. For example, as shown in FIG. 3, beyond the distal portion 24, the secondary mandrel 2b may be moved to position A3 to allow the secondary mandrel 2b to exit from the tubular body 10, e.g., to facilitate attachment to the pull ring 38, as described further elsewhere herein.

Finally, in position A4, the secondary mandrel 2b may be received within a feature of the primary mandrel 2a, thereby positioning the secondary mandrel 2b within the primary lumen 18a, as shown in FIG. 5C. For example, the primary mandrel 2a may include an enclosed lumen, open groove, or other channel 3a extending along a length of the primary mandrel 2a corresponding to the intermediate portion 22 of the catheter 10, and the secondary mandrel 2b may be received within the channel 3a to allow the liner 40 and reinforcement members 42 to be applied around the primary mandrel 2a while the secondary mandrel 2b remains within the channel 3a, thereby omitting an auxiliary lumen along the section.

Optionally, as shown in FIG. 5A, the jacket source 90 may be provided for applying one or more layers of jacket material around the reinforcement-wrapped mandrels 2. For example, a co-extruder, laminator, or other applicator may be provided that applies melted, uncured, and/or otherwise raw jacket material 7, e.g., from a hopper or other container (not shown), or rolls sheets of jacket material 7 may be wrapped around the reinforcement members 43 and mandrels 2. Exemplary materials for the jacket material 7 include plastics, e.g., thermoplastics, such as polyether block amide, nylon, or urethanes, thermoset plastics, metals, or composite materials. Alternatively, other processing may be used to bond or otherwise attach the jacket material 7 to the liner material 4 and/or embed the reinforcement members 43 in the jacket material 7, thereby resulting in an integral tubular body 9. The resulting tubular body 9 (with or without jacket material 7) may be collected, e.g., on a capture reel or in a container (not shown).

Thereafter, the tubular body 9 may be further processed to make a device, such as the apparatus 8 shown in FIGS. 1A and 1B. Other components may be added to the individual tubular devices, as desired for the particular application, as described elsewhere herein.

Turning to FIG. 6, an exemplary method is shown for making a single-piece tubular body, such as the tubular body 11 shown in FIG. 3. To facilitate making the tubular body 11, a primary mandrel 102 may be provided that has a variable cross-section along its length corresponding to different portions or sections of the tubular body 11. The variable cross-section may allow reinforcement members to be braided substantially continuously along the length of the primary mandrel 102, e.g., to secure liner material (not shown) around the mandrel 102, while adding, moving, and/or removing one or more secondary mandrels along one or more sections of the primary mandrel 102.

For example, as shown in FIG. 6, the primary mandrel 102 may have a first section 112 and a second section 114 having different dimensions. In the example shown, the first section 112 has a substantially uniform cross-section along its length from a first end 102a of the primary mandrel 102 to the second section 114. For example, as shown in FIG. 6A, the first section 112 may define a substantially uniform diameter C1 along its length, although alternatively, the first section may have an oval or other non-circular cross-section (not shown), if desired. The first section 112 may have a length corresponding to an intermediate and/or proximal portion of a tubular body, e.g., including at least the intermediate portion 22 of the tubular body 10 shown in FIGS. 1A and 1B.

The second section 114 may have a cross-section that is smaller than the first section 112. For example, as shown in FIG. 6A, the second section 114 may have a cross-sectional dimension C2 that is smaller than the diameter C1 (in the same direction transverse to the longitudinal axis 120. In the example shown, the second section 114 may include a pair of substantially flat surfaces 116 extending along the length of the second section 114 and spaced apart to define dimension C2. Consequently, a step-down or other transition region 113 may be provided between the first and second sections 112, 114 that defines a pair of end walls 115 transitioning between the two cross-sections C1, C2. The width of the second section 114 (orthogonal to the dimension C2) may be the same or smaller than the dimension C1.

In addition, one or more lumens, grooves, or other channels may be provided in the primary mandrel 102 for receiving at least a portion of one or more secondary mandrels. For example, as shown, each end wall 115 may include an opening communicating with an enclosed lumen 118 extending substantially parallel to a longitudinal axis 120 of the primary mandrel 102. Each lumen 118 may only extend a relatively short distance axially from the end wall 115 into the first section 112, e.g., along intermediate section 113. Alternatively, each channel may extend the entire length of the first section, if desired (not shown).

During assembly, the first end 102a of the primary mandrel 102 may be fed into a braiding apparatus, e.g., through the guide 66 of the braiding apparatus 60 shown in FIG. 5A, and a plurality of reinforcement members 43 may be braided or otherwise wrapped around the first section 112 of the primary mandrel 102, e.g., over a liner (not shown), as described elsewhere herein. As the reinforcement members 43 approach the transition region 113, a first end 104a of a secondary mandrel 104 may be inserted into each lumen 118, e.g., such that the secondary mandrels 104 extend along the second section 114, as shown in FIG. 6. For example, the secondary mandrels 104 may extend from a passage 73a through one of the horn gears 72a in the braiding apparatus 60, i.e., such that the secondary mandrels 104 are provided at the A1 position shown in FIG. 5B adjacent the second section 114. When the reinforcement members 43 are then wrapped around the second section 114, the secondary mandrels 104 may be included in the resulting braid such that some reinforcement members 43 pass over and under the secondary mandrels 104 along the second section 114.

Consequently, the reinforcement members 43, and any liner surrounding the primary mandrel 102 (not shown in FIG. 6 for clarity), may follow the contour of the primary mandrel 102 resulting in a transition between the first and second sections 112, 114, e.g., corresponding to the transition and end wall 21 between the first and second segments 18a1, 18a2 of the primary lumen 18a shown in FIG. 3.

If desired, beyond the second section 114, the secondary mandrels 104 may be moved to position A3 shown in FIG. 5B, e.g., such that the secondary mandrels 104 are outside the braid of the reinforcement members 43 to provide an extension 27, as shown in FIG. 3. Such positioning may provide an exit opening 23 from the auxiliary lumen 18b, e.g., to facilitate attaching a steering element 36 to a pull ring 38 positioned over the extension 27, e.g., as shown in FIG. 3, and as described further herein.

Optionally, an outer layer may be applied around the braided mandrel assembly during or immediately after the braiding operation using the apparatus 60, if desired, or the braided assembly may be processed further separate from the braiding apparatus 60. For example, as shown in FIG. 3, an outer layer 44 may be applied around the reinforcement members (not shown) to provide the wall of the intermediate and distal portions 22, 24. At any time, the reinforcement members 43 beyond the second section 114 of the primary mandrel 102 may be cut or otherwise severed to provide a braided assembly that may be subsequently processed.

In one example, to make a braided assembly similar to the example shown in FIG. 4, the primary and secondary mandrels 102, 104 may be removed after the braiding process shown in FIG. 6, and then a steering element 36 may be introduced into the braided assembly, e.g., through the resulting primary lumen 18a and into each of the auxiliary lumens 18b. A pull ring 38 may be positioned adjacent the distal portion 24, e.g., over the extension 27 and distal ends 36b of the steering elements 36 may be attached to the pull ring 38. Each steering element 36 may have sufficient length such that a proximal end 36a thereof extends from the proximal end of the braided assembly, e.g., for connection to an actuator within a handle (not shown), similar to examples described elsewhere herein.

A distal end 54 of a compression-resistant member 50 may be inserted into the primary lumen 18a over each steering element 36 and advanced until the distal end 54 is positioned adjacent the end wall 21 and/or other transition. The compression-resistant member 50 may have sufficient length such that a proximal end 52 thereof extends from the proximal end of the braided assembly, e.g., for positioning adjacent a stop within a handle (not shown), also similar to examples described elsewhere herein.

Alternatively, the secondary mandrel(s) is used as a steering element rather than adding a steering element after removing the secondary mandrel(s) 104 shown in FIG. 6. In this alternative, the primary mandrel 102 may include a channel extending the length of the first section 112 (not shown) for receiving each secondary mandrel steering element such that the secondary mandrel is positioned inside the reinforcement members and liner along the first section 112. The secondary mandrel may be positioned at location A1 at the transition to the second section such that the secondary mandrel is braided into the reinforcement members, as described previously. Optionally, a tubular member (not shown) may be positioned around the secondary mandrel having sufficient length to extend along the distal portion to define the inner diameter of the auxiliary lumen 18b. The tubular member may be removed at any time after assembly to provide space around the secondary mandrel to allow the secondary mandrel to slide freely within the auxiliary lumen.

The distal end of the secondary mandrel may be positioned beyond the distal portion to allow the distal end to be attached the pull ring, thereby providing a steering element. When the primary mandrel 102 is removed, the length of the secondary mandrel within the channel may simply slide along the channel as the primary mandrel 102 is pulled, thereby releasing the secondary mandrel within the resulting primary lumen 18a. The length of the secondary mandrel may be sufficient to extend from the resulting tubular member, again for connection to an actuator. A compression-resistant member may then be advanced over the secondary mandrel, now steering element, into the primary lumen, similar to the previous methods.

FIG. 4 shows an example of a tubular body 9 that may result from these methods. The tubular body 9 may include an outer layer (not shown for clarity) if the outer layer is applied before removing the primary mandrel 102 (and secondary mandrel(s) if separate steering element(s) are added). Alternatively, if the tubular body 9 is assembled without an outer layer, i.e., with liner (not shown), reinforcement layer 42, steering element(s) 36, compression-resistant member(s) 50, and pull ring 38, an outer layer may be applied subsequently, as desired.

The resulting tubular body 9 may be incorporated into a finished device, such as the apparatus 8 shown in FIGS. 1A and 1B, by adding one or more additional components for the outer layer, if desired, adding one or more tip segments (not shown, e.g., over the pull ring 38, and the like. Additional components, e.g., the handle 50 and its components, may then be added to the assembly, e.g., attached to the proximal end of the tubular body 9, as desired. Optionally, other components, e.g., one or more sensing elements, electrodes, balloons, and the like (not shown), may be added to the catheter as desired.

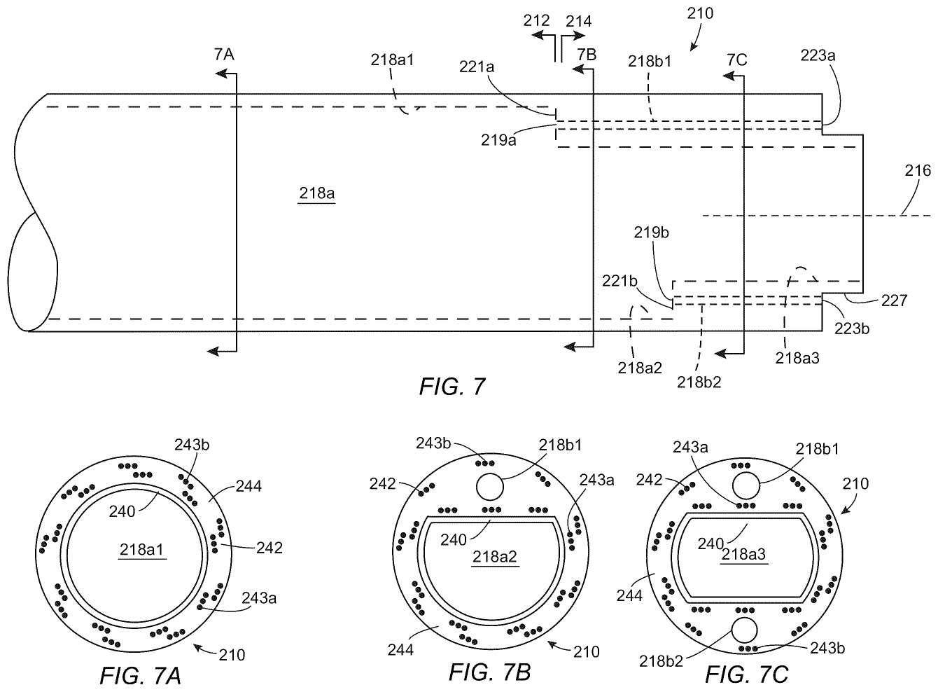

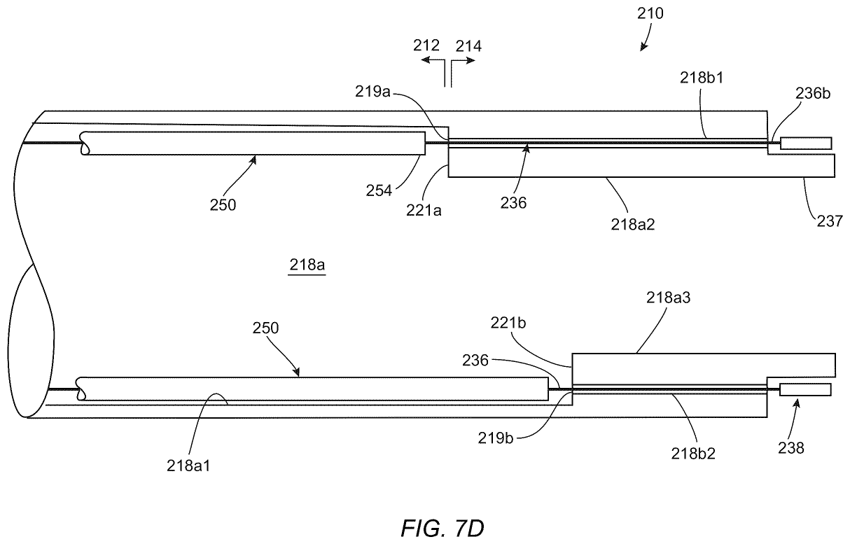

Turning to FIG. 7, another example of a tubular body 210 is shown that is formed from a single-piece construction including different portions or sections having different cross-sections and/or composition between first and second ends of the tubular body 210. For example, as shown, the tubular body 210 includes a first portion 212, e.g., corresponding to intermediate and/or proximal portions 22, 20 of a catheter shaft of the apparatus 8 shown in FIGS. 1A and 1B, and a second portion 214, e.g., corresponding to a steerable distal portion 24, extending axially along a longitudinal axis 216 of the tubular body 210. The first portion 212 may extend from the first end of the tubular body 210 (not shown to the left in FIG. 7) axially to a first end wall or transition 221a. Along the first portion 212, the tubular body 210 includes a single lumen, i.e., a first segment 218a1 of the primary lumen 21a. The first segment 218a1 may have a substantially circular cross-section, as shown, or other desired cross-section, which may be substantially uniform or variable along the length of the first portion 212, similar to other examples herein.

The first portion 212 may be constructed similar to other examples herein, e.g., including an inner liner 240 surrounding the primary lumen 18a, a reinforcement layer 242 including a plurality of reinforcement members 243 braided around the liner 240 along the length of the first portion 212, and an outer jacket or layer 244.

The second portion 214 includes a second segment 218a2 of the primary 218a communicating with the first segment 218a1 and extending along the longitudinal axis 216 towards the second end of the tubular body 210 (e.g., ending at extension 227). The second portion 214 includes a first auxiliary lumen 218b1 extending along the second portion 214 adjacent the second segment 218a2 from a first or proximal opening 219a at a first end wall or other transition 221a between the first and second portions 212, 214 towards the extension 227. For example, the first auxiliary lumen 218b1 may extend from the proximal opening 219a to a second or distal opening 223a adjacent the extension 227.

In addition, unlike the previous examples, the second portion 214 includes a second auxiliary lumen 218b2 extending from a second end wall or transition 221b adjacent the second segment 218a2 towards the extension 227, e.g., to a second or distal opening 223b adjacent the extension 227. As shown, the second end wall 221b is spaced axially from the first end wall 221a, e.g., closer to the extension 227 than the first portion 212. The second auxiliary lumen 218b2 may be offset circumferentially from the first auxiliary lumen 218b1, e.g., about one hundred eight degrees (180°) around the longitudinal axis 216. The second auxiliary lumen 218b2 may have an axial length shorter than the first auxiliary lumen 218b1, although the second openings 223a, 223b may be axially aligned adjacent the extension 227. Consequently, steering elements (not shown) introduced through the auxiliary lumens 218b1, 218b2 may be coupled to the same pull ring or other tip (not shown) attached to the extension 227, similar to other devices herein.

As best seen in FIG. 7C, along the distal portion 214 including the second auxiliary lumen 218b2, the primary lumen 218a may include a third segment 218a3 smaller than the second segment 218a2 (shown in FIG. 7B), which is, in turn, smaller than the first segment 218a1 within the first portion 212 of the tubular body 210. Along the second portion 214, the construction of the wall of the tubular body 210 may be similar to other devices herein, e.g., including an inner liner 240 surrounding the primary lumen 218a, a reinforcement layer 242, and an outer jacket or layer 244. In the example shown, the auxiliary lumens 218b1, 218b2 may extend through the reinforcement layer 242, e.g., braided into the reinforcement layer 242 such that some reinforcement members 243a only surround the primary lumen 218a while some reinforcement members 243b surround both the auxiliary lumens 218b1, 218b2 and the primary lumen 218a, similar to other devices described herein and in the references incorporated by reference herein.

Consequently, similar to other tubular bodies described elsewhere herein, the tubular body 210 may include a continuous braid of reinforcement members 243 wrapped around both the first and second portions 212, 214 such that the tubular body 210 is formed as a single-piece including the lumens 218a, 218b therein. As with other tubular bodies herein, steering elements, compression-resistant members, pull rings, tips, and/or other components (not shown) may be added to the tubular body 210 to provide a finished device, such as the apparatus 8 shown in FIGS. 1A and 1B.

For example, as shown in FIG. 7D, a steering element 236 may be received through the first segment 218a1 of the primary lumen 218a through the opening 219a, 219b into the respective auxiliary lumens 218b1, 218b2, and distal ends 236b of the steering elements 236 may be attached to a pull ring 238 mounted over the extension 227, similar to other devices herein. In addition, compression-resistant members 250 may be positioned over each steering element 236 with their distal ends 254 abutting or otherwise positioned adjacent the end walls 221a, 221b.

Optionally, the liner 240 of the tubular body 210 may electrically insulate elements or devices within the primary lumen 218a from components of the tubular body 210 outside the liner 240. PTFE or FEP may be optimal materials for the liner 240 because of their excellent dielectric strength and low friction. Alternatively, in such applications, PEBAX, urethane, or other thermoplastic materials may provide greater integrity and encapsulation of the braid than fluoropolymers.

To make the tubular body 210 shown in FIG. 7, a bilaterally stepped primary

mandrel may be used, i.e., that includes multiple stepped-down or transition regions spaced axially apart from one another to create the auxiliary lumens 218b1, 218b2 that include proximal openings 219a, 219b that are axially offset from one another, resulting in a “double D” shaped primary lumen. Such construction may allow compression coil stops, e.g., end walls 221a, 221b to be formed and located at different axial positions such that, steering elements (not shown) received in each auxiliary lumen 218b1, 218b2 may be coupled to the same pull ring. The resulting steering element configurations may result in different deflection section lengths during activation of the respective steering elements and thus different bend radii/diameters.

Such a configuration may provide multiple or more complicated curvatures in a steerable portion of the resulting apparatus, which may be desirable in numerous circumstances where the range in encountered anatomy and or relation to the access site requires different amounts of reach and curve diameter. The asymmetry may also allow for a longer reach and/or larger curve (for a given deflection angle) on one side (actuating one steering element) and a shorter curve on the other side (actuating the other steering element). For example, accessing from the interatrial septum, the desired reach for the right pulmonary veins may be different than for the left pulmonary veins that benefit from longer reach. In the left ventricle when accessing through the mitral valve, the ventricle wall is closer to the mitral valve on one side than the other. Asymmetric deflection may allow an operating physician or other user the ability to use one curve when needed and the other when needed all in the same case with the same device.

Alternatively, symmetric deflection may be easily achieved as well where the end walls/stops are formed and/or located at the same axial position. It is noted the more than two stops/pull wires may also be implemented in order to achieve, for example, deflection in multiple directions, using similar constructions and methods as described above.

In the example shown in FIG. 7, the primary lumen 218a may have a “double D” cross-section along the second portion 214. For example, along the portion only including the first auxiliary lumen 218b1 (shown in FIG. 7B), the second segment 218a2 of the primary lumen 218a may generally define a “D” shaped cross-section including a substantially straight or flat wall adjacent the auxiliary lumen 218b1 and a semi-circular or other curved wall opposite the flat wall, e.g., following the outer curve of the tubular body 210. Along the portion including both first and second auxiliary lumens 218b1, 218b (shown in FIG. 7C), the third segment 218a2 of the primary lumen 218a may include a generally rectangular cross-section including substantially flat walls adjacent both auxiliary lumens 218b1, 2182 and curved walls between the flat walls.

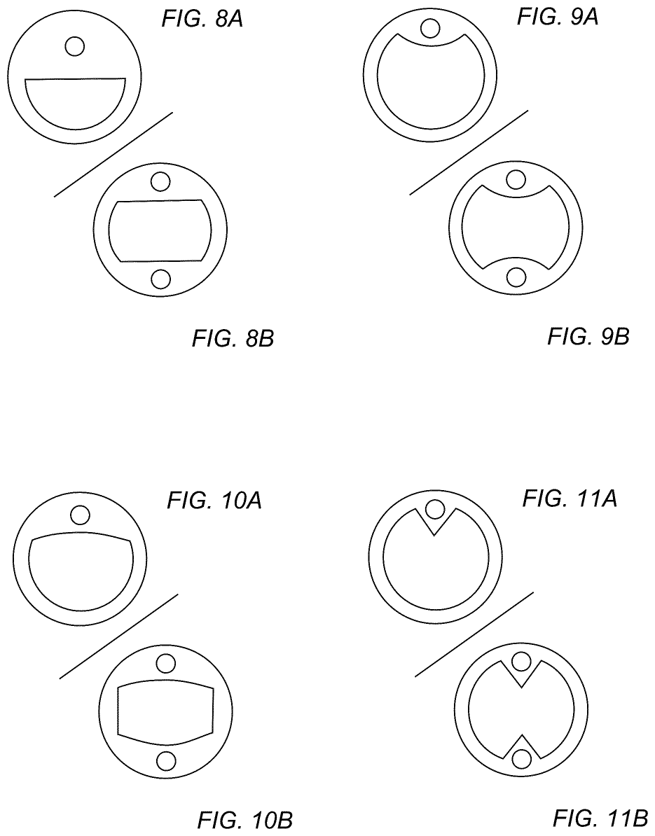

FIGS. 8A-11B show variations of cross-sections that may be provided in the primary lumen along a portion including one or two auxiliary lumens on opposite sides of the primary lumen. For example, in FIGS. 9A and 9B, the primary lumen may include a concave wall adjacent one or both auxiliary lumens, while in FIGS. 10A and 10B, the primary lumen may include convex walls adjacent one or both auxiliary lumens. Alternatively, in FIGS. 11A and 11B, the primary lumen may include “V” shaped walls adjacent each of the auxiliary lumens, thereby changing the shape of the primary lumen as it passes through the portion including one or both auxiliary lumens. The shape of the inner wall(s) of the primary lumen may facilitate centering a steering element passing through the primary lumen and/or may increase space within the tubular body for receiving other components of the finished device, e.g., one or more wires, cables, and the like (not shown).

Turning to FIGS. 14A-14C, an exemplary pull ring 438 is shown that may mounted to a distal end of a tubular device, such as any of the devices described elsewhere herein, which may be coupled to a single elongate wire or cable to provide a pair of steering elements 436. For example, the pull ring 438 may be attached over or to an extension beyond a steerable portion of a tubular body, e.g., distal to the distal portion 24 of the tubular body 11 shown in FIG. 3. In the example, shown, a single cable is provided for both steering elements 436 with an intermediate portion of the cable received through and/or attached to the pull ring 438. The length of the cable may be sufficient such that the two ends may pass through respective auxiliary lumens 18b and primary lumen 18a, e.g., similar to the separate steering elements 36 shown in FIG. 3. For example, the intermediate portion of the cable may be received through recesses in the pull ring and then secured, e.g., by one or more of crimping, bonding with adhesive, welding, and the like.

The foregoing disclosure of various examples has been presented for purposes of illustration and description. It is not intended to be exhaustive or to limit the invention to the precise forms disclosed. Many variations and modifications of the embodiments described herein will be apparent to one of ordinary skill in the art in light of the above disclosure.

Further, in describing representative embodiments, the specification may have presented the method and/or process as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims.

While the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents and alternatives falling within the scope of the appended claims.

1. An apparatus for performing a procedure within a patient's body, comprising:
a tubular member comprising a proximal end, a distal end sized for introduction into a patient's body, a longitudinal axis extending between the proximal end and the distal end, an intermediate portion extending partially between the proximal end and the distal end, and a distal portion extending distally from the intermediate portion at a transition to the distal end;
a primary lumen extending between the proximal end and the distal end;
an auxiliary lumen extending along the distal portion adjacent the primary lumen from an opening communicating with the primary lumen at the transition to the distal end;
a steering element comprising a first portion slidably disposed within the auxiliary lumen and terminating at a first end fixed to the distal end distally beyond the auxiliary lumen and a second portion passing proximally through the opening into the primary lumen and extending to a second end adjacent the proximal end;
an actuator on the proximal end coupled to the second end of the steering element such that, actuation of the actuator applies axial tension or compression to the first portion of the steering element, thereby causing the distal portion to bend; and
a compression-resistant member disposed around the first portion of the steering element within the primary lumen between the transition and the proximal end for preventing forces from the steering element from transferring to the tubular member proximal to the distal portion.

2. The apparatus of claim 1, wherein the primary lumen has a first segment along the intermediate portion defining a first internal diameter or cross-sectional dimension and a second segment along the distal portion defining a second internal diameter or cross-sectional dimension smaller than the first segment.

3. The apparatus of claim 2, wherein the primary lumen comprises an end wall at the transition transitioning from the first segment to the second segment.

4. The apparatus of claim 3, wherein the end wall extends substantially perpendicular to the longitudinal axis.

5. The apparatus of claim 2, wherein the second segment communicates with an outlet in a distal tip of the distal end.

6. The apparatus of claim 1, wherein the intermediate portion comprises a plurality of reinforcement members comprising windings wound helically within a wall of the intermediate portion around the primary lumen, and wherein the distal portion comprises a plurality of reinforcement members comprising windings wound helically within a wall of the distal portion such that at least some of the windings pass between the primary lumen and the auxiliary lumen and at least some of the windings surround both the primary lumen and the auxiliary lumen.

7. The apparatus of claim 6, wherein the plurality of reinforcement members are braided continuously along the intermediate and distal portions.

8. The apparatus of claim 6, wherein, beyond the distal portion, the first end of the steering element is positioned radially outside the plurality of reinforcement members.

9. The apparatus of claim 8, further comprising a pull ring fixed to the distal end beyond the distal portion and wherein the first end of the steering element is attached to the pull ring.

10. An apparatus for performing a procedure within a patient's body, comprising:
a tubular body comprising a proximal end, a distal end sized for introduction into a patient's body, a longitudinal axis extending between the proximal end and the distal end, an intermediate portion extending partially between the proximal end and the distal end, and a distal portion extending distally from the intermediate portion at a transition to the distal end;
a primary lumen extending between the proximal end and the distal end, the primary lumen defining a first segment along the intermediate portion defining a first internal diameter or cross-sectional dimension and a second segment along the distal portion defining a second internal diameter or cross-sectional dimension smaller than the first segment;
an auxiliary lumen extending along the distal portion adjacent the primary lumen from an opening at the transition communicating with the primary lumen at the transition to the distal end;
a steering element comprising a first portion slidably disposed within the auxiliary lumen and terminating at a first end fixed to the distal end distally beyond the auxiliary lumen and a second portion passing proximally through the opening into the primary lumen and extending to a second end adjacent the proximal end;
an actuator on the proximal end coupled to the second end of the steering element such that, actuation of the actuator applies axial tension or compression to the first portion of the steering element, thereby causing the distal portion to bend;
a compression-resistant member disposed around the first portion of the steering element within the primary lumen between the transition and the proximal end for preventing forces from the steering element from transferring to the tubular member proximal to the distal portion; and
a plurality of reinforcement members braided continuously along the intermediate and distal portions, wherein, along the distal portion, the plurality of reinforcement members comprising windings wound helically within a wall of the distal portion such that at least some of the windings pass between the primary lumen and the auxiliary lumen and at least some of the windings surround both the primary lumen and the auxiliary lumen.

11. The apparatus of claim 10, wherein the primary lumen comprises an end wall at the transition transitioning from the first segment to the second segment.

12. The apparatus of claim 11, wherein the end wall extends substantially perpendicular to the longitudinal axis.

13. The apparatus of claim 10, wherein, beyond the distal portion, the first end of the steering element is positioned radially outside the plurality of reinforcement members.

14. The apparatus of claim 10, wherein the auxiliary lumen is a first auxiliary lumen, the tubular body further comprising a second auxiliary lumen extending along the distal portion adjacent the primary lumen from a second opening at a second transition towards the distal end.

15. The apparatus of claim 3, wherein a distal end of the compression-resistant member abuts the end wall.

16. The apparatus of claim 15, wherein the distal end of the compression-resistant member is free-floating on the steering element immediately adjacent the end wall.

17. The apparatus of claim 16, wherein a radial dimension of the end wall is smaller than a diameter of the distal end of the compression-resistant member such that the distal end of the compression-resistant member only partially contacts the end wall.

18. The apparatus of claim 16, further comprising a stop adjacent the proximal end of the tubular member for preventing a proximal end of the compression-resistant member from moving proximally.

19-32. (canceled)

33. A single-piece tubular body for a medical comprising, comprising:
a first portion including a first primary lumen segment extending from a first end of the tubular body along a longitudinal axis of the first portion;
a second portion adjacent the first portion including a second primary lumen segment communicating with the first primary lumen segment and extending along the longitudinal axis towards a second end of the tubular body, and an auxiliary lumen extending along the second portion adjacent the second primary lumen segment from an opening at a transition between the first and second portions towards the second end; and
a plurality of reinforcement members braided continuously along the first and second portions.

34-48. (canceled)

49. A method for making a tubular body, comprising:
providing a primary mandrel comprising a first section defining a first cross-sectional dimension and a second portion defining a second cross-sectional dimension smaller than the first cross-sectional dimension;
braiding reinforcement members around a liner along the first section towards the second section;
positioning a secondary mandrel outside the primary mandrel adjacent the second section;
braiding the reinforcement members along the second section such that the secondary mandrel is included within a braid of the reinforcement members to define an auxiliary lumen;
applying an outer jacket around the primary mandrel after braiding the reinforcement members around the first and second sections; and
removing the primary mandrel to define a primary lumen including a first segment corresponding to the first section of the primary mandrel, defining a first portion of the tubular body, and a second segment corresponding to the second section of the primary mandrel, defining a second portion of the tubular body, the auxiliary lumen extending along the second portion adjacent to the second segment.

50-54. (canceled)
